(19) United States
(12) Patent Application Publication
(10) Pub. No.: US 2024/0066073 A1
(43) Pub. Date: Feb. 29, 2024

(54) READILY DISPERSIBLE SHELF-STABLE MICROBIAL GRANULES

(71) Applicant: Danisco US Inc., Palo Alto, CA (US)

(72) Inventors: Nathaniel T Becker, Burlingame, CA (US); Samuel A Maurer, San Francisco, CA (US); Jared Sindelar, Santa Cruz, CA (US); Henricus Jacobus Cornelis Gommeren, Los Altos, CA (US); Ashwin Bala, Palo Alto, CA (US)

(21) Appl. No.: 18/247,965

(22) PCT Filed: Oct. 6, 2021
(86) PCT No.: PCT/US2021/053741

§ 371 Date: Apr. 5, 2023

Related U.S. Application Data
(60) Provisional application No. 63/088,272, filed on Oct. 6, 2020.

(51) Int. Cl. A61K 35/74; A61K 9/16; A61K 36/064; A61K 35/747; A61K 36/066; A61K 35/745; A01N 25/26; A01N 63/30; A01N 63/20

(52) U.S. Cl. CPC A61K 35/74; A61K 9/1676; A61K 36/064; A61K 9/1623; A61K 9/1652; A61K 9/1611; A61K 35/747; A61K 36/066; A61K 9/1641; A61K 35/745; A01N 25/26; A01N 63/30; A01N 63/20

(57) ABSTRACT

Described are compositions and methods relating to layered granules comprising a core surrounded by a coating layer containing bioactive agents distributed within a protectant matrix, wherein the protectant matrix includes at least one polyhydroxy compound and at least one phosphate compound. The core and the protectant matrix are water soluble and fast-dissolving.

### CROSS-REFERENCE OF RELATED APPLICATIONS

This application is a 371 of International Application No. PCT/US2021/053741, filed Oct. 6, 2021, and claims the benefit of U.S. Provisional Application No. 63/088,272, filed Oct. 6, 2020, all of which are hereby incorporated by reference in their entirety.

### FIELD OF THE INVENTION

The described compositions and methods relate to stabilizing and delivering bioactive agents such as microorganisms and enzymes in a readily dispersible layered granule. The layered granule comprises a core surrounded by a coating layer that includes at least one bioactive agent distributed within a protectant matrix, wherein the protectant matrix includes at least one polyhydroxy compound and at least one phosphate compound, and wherein the core is water-soluble and fast-dissolving.

### BACKGROUND

In delivery of beneficial bioactive agents such as microorganisms and enzymes to applications such as agriculture, animal husbandry, and human health, there is a need for shelf stable solid formulations that readily dissolve or disperse in water or aqueous media, within about a minute or less, with minimal agitation, and without producing insoluble residues that could settle in a container, or clog screens, filters or nozzles used in spray and irrigation systems. A shelf stable bioactive formulation should maintain viability and efficacy for at least one month, preferably for at least 6 months or longer, at ambient temperature and humidity, without the need to reduce storage humidity or water activity to very low levels, e.g., less than about 20% RH or 0.20 aw, by measures such as freeze drying, desiccants, or special packaging. Solid formulations are furthermore preferably towable and non-dusty. Granules are preferred over fine powders, which tend to be dusty or to clump when dispersed in water.

While some enzymes are inherently thermostable, many effective enzymes, otherwise suitable for use in industrial or therapeutic applications, exhibit poor thermostability, oxidative stability, or are otherwise susceptible to loss in activity during storage. Stabilization can often be improved by means of formulation as stable liquids or granules. Shelf-stable enzyme granules are well known in the art. Enzyme granules produced by means of different technologies, exhibiting excellent retention of enzymatic activity during storage at ambient or super-ambient temperature and humidity, without the use of specialized packaging or desiccants have found use in laundry detergents, animal feed, textile processing etc.

Enzyme granulation processes and formulations useful for imparting improved storage stability include fluidized-bed granulation, high-shear granulation, extrusion and Oiling. These technologies are described in the literature [1, 2]. For example, WO1993/07263 describes a coated enzyme granule exhibiting improved stability and delayed release characteristics, comprising a core, an enzyme layer and an outer layer, wherein at least one layer comprises a. vinyl polymer such as polyvinyl alcohol. The granules exhibit excellent stability during storage in laundry detergents. WO2007/44968A2 describes stable durable granules with active agents, such as enzymes, comprising a core, at least one active agent, and at least one coating. The described granules demonstrate excellent stability during ambient storage and also survive steam pelleting in animal feed. WO1989/08694 describes an enzyme granulate comprising an enzyme containing core with a coating containing a mono- and/or di-glyceride of a fatty acid, applied in a rotating mixer. Due to the coating the enzyme stability is enhanced in detergents, even detergent comprising strong bleaching agents.

However, the above-cited technologies and formulations for producing stable enzyme granules while advantageous for protecting and stabilizing enzymes, employ coatings, binders or excipients one or more slow-dissolving high molecular weight polymers such as polyvinyl alcohol, hydrophobic compounds such as mono and di-glycerides, insoluble pigments such as titanium dioxide, aid/or insoluble mineral processing aids and barrier materials, such as talc, any of which are prone to dissolve or disperse slowly, or leave residues that settle or can plug or foul screens, filters and nozzles upon dispersion in water.

Certain microbial structures of microorganisms are inherently stable, such as robust vegetative cells, or spores. U.S. Pat. No. 5,929,507 describes a composition suitable for stabilizing microorganisms for use as plant seed inoculants, by combining the microorganisms with soluble non-cross-linked polysaccharides such as alginate. The utility of alginate encapsulation is limited to thermally stable microbial structures.

Many other beneficial microorganisms are sensitive to temperature, moisture, and oxygen, and other environmental stressors, and hence tend to lose viability quickly, sometimes within hours or days. It is generally known that the shelf life of viable microorganisms can be ensured or improved by storage under refrigeration, in a dry format, at low water activity, or in packaging with controlled humidity and permeability. Water activity of the microorganisms can be reduced to low levels, e.g., below 0.4, 0.2 or even below 0.1, by drying processes such as lyophilization or freeze drying, generally in combination with addition of protectant stabilizers. U.S. Pat. No. 9,469,835 describes a means of stabilizing a variety of microorganisms, including bacteria and fungi, by a vitrification process. The patent described a two-step drying process, first under vacuum from a partially-frozen slush state at near subzero temperatures, followed by drying at temperatures above 40° C. until a very low water content and low water activity is achieved. This process is asserted to produce a mechanically stable foam with significantly elevated glass transition temperature (T_{g}) such that the biological is at no time subjected to temperatures as high as the T_{g}.

U.S. Patent Pub. No. 20140004083 discloses a cryoprotectant system for preserving microorganisms, such as lactic acid bacteria, by addition of a non-reducing sugar, such as trehalose, combined with inositol in a specified range of ratios, and thereafter freezing, vacuum drying, or freeze-drying the composition for dry storage. U.S. Pat. No. 9,308,271 describes a cryoprotectant system for preserving lactic acid bacteria by addition of trehalose, inulin, and hydrolyzed casein, in the absence of alginate, thereafter freeze-drying the composition and storing the composition at up to 35° C. and at a water activity of 0.3 or less. In such cases where a microbial composition has been freeze-dried or vacuum-dried, low water activity can be maintained by packaging the microbial composition within capsules, bottles, or pouches composed of barrier materials with low water vapor permeability.

Some attempts have been made to provide compositions that preserve the viability of microbes during storage at ambient temperature and humidity without freeze drying or special packaging. U.S. Patent Pub. No. 2012014253 describes a composition and process for stabilizing dehydrated microorganisms by producing a core particle comprising dried organisms and coating the core particle with hygroscopic salts, such as a mixture of phosphate salts. The hygroscopic salt coating in effect acts as a water decoy, attracting moisture from the environment and trapping it before it can diffuse into the dehydrated microorganisms in the core particle. While the examples provide some evidence of stability enhancement during storage at moderate humidity conditions, the hygroscopic salt layer eventually saturates with absorbed water and ultimately loses its ability to protect moisture-sensitive microorganisms from taking up water and losing viability. Thus, there remains a need for a robust and sustained way to preserve the viability of microorganisms stored at ambient temperature and humidity conditions, without the need for special packaging or other measures to refrigerate and maintain low water activity of the microorganisms.

For example, in agricultural crop applications, it would be desirable to provide bioactive products intended to stimulate plant growth or control plant pests and diseases formulated dry formulations that can be stored without refrigeration or humidity resistant packaging in an open environment, and then applied as needed by first solubilizing in aqueous solution and then applied to the crops via foliar spray or drip irrigation.

Typical dry formulations for use in agriculture include wettable powders and granules that can be applied directly in the field, or first diluted to form solutions or suspensions that can be sprayed onto plants. However, such powders and granules are often formulated with insoluble materials such as clay, silica, mica, talc, cellulose fibers and the like, or with high molecular weight natural or synthetic polymers and binders, such as alginate or polyvinyl alcohol, resulting in particles that disperse or dissolve slowly or incompletely. Such incompletely soluble or slowly dispersing granules and powders tend to leave insoluble residues that settle in tanks or clog screens, filters and nozzles commonly used in agricultural spray and irrigation systems. Wettable powders are often difficult to disperse in water, forming lumps upon wetting, and tend to exhibit poor flowability and dustiness. Dusts in agriculture are problematic as they can represent an inhalation hazard and may require specialized personal protectant equipment to ensure worker safety.

In animal husbandry applications, beneficial microbes can improve health or protect against disease. It is desirable to provide a stable, dry form of the beneficial microbes that will remain viable for weeks or months during storage at ambient temperatures and humidity, without special packaging or refrigeration, prior to introducing the microbes into drinking water, or alternatively, mixing into dry animal feed mash composed of corn or soy grain with significant free water content. A dry formulation for introducing beneficial microbes into drinking water should dissolve quickly, with minimal agitation, and without leaving behind insoluble residues that could settle in the water line or plug drinker nozzles.

Beneficial microorganisms are also provided as probiotics to promote human health and prevent or combat disease. Probiotic supplements are often provided in a dry form, either as powder or in unit dose forms as sachets or capsules. Regardless of the particular format, probiotic supplements are packaged in sealed containers with controlled humidity, often maintained by the use of desiccant packs, and typically with recommendations to store either refrigerated or in a cool, dry place. For supplements taken by adding a powder or other dry form to water or beverages, fast dissolution and lack of clumping would be advantageous.

For food applications, microorganisms are often incorporated to provide probiotic benefits. In moist or foods such as snack bars, the incorporated microorganisms must remain viable for several months or longer under ambient conditions. Probiotic organisms are also incorporated into pet foods such as kibbles. In these applications, there is often no provision for refrigerated storage or special packaging to protect against humidity.

Microorganisms suitable as beneficial agents in the applications described above include any bacteria, yeast or fungi, and can comprise multiple different cell morphologies, including vegetative cells, mycelia, spores, or cysts. Depending on the particular species, certain of these forms may have significantly enhanced storage stability. For example, many bacterial and fungal species produce spores that retain viability during extended periods of dormancy, for months or even years, until re-activated under germination conditions.

However, many microbial structures do not remain viable under ambient conditions, i.e. temperatures above about 10° C., most typically 20-30° C., and humidity above about 10% RH, most typically 30-60% RH, for extended storage periods, i.e., longer than one day, most typically 3-52 weeks. For example, many vegetative bacterial cells, fungal spores, and fungal microsclerotia do not show extended viability at these conditions.

There is a need for shelf-stable formulations for bioactive agents such as microorganisms and enzymes, and processes for producing such formulations, that ensure improved viability and stability of bioactive agents during storage under ambient conditions, e.g., at conditions that do not require refrigeration, humidity control or special packaging, and which simultaneously provide a convenient product form that dissolves or disperses rapidly, e.g., in less than one minute with minimal agitation, and does not leave residues that settle in tanks or clog screens, filters and nozzles commonly used in industrial applications.

### SUMMARY

Described are compositions and methods relating to coated granules consisting of a core surrounded by a coating layer containing at least one bioactive agent distributed within a protectant matrix that includes at least one polyhydroxy compound and at least one phosphate compound. The core and protectant matrix are water-soluble and fast-dissolving.

Aspects and embodiments of the granules are described in the following, separately-numbered paragraphs.

1. In one aspect, a layered granule comprising a core surrounded by a coating layer comprising at least one bioactive agent distributed within a protectant matrix, wherein the protectant matrix comprises: (a) at least one polyhydroxy compound; and (b) at least one phosphate compound; and wherein the at least one polyhydroxy compound, the at least one phosphate compound, and the core are water soluble and fast-dissolving.

2. In some embodiments of the granule of paragraph 1, the core has a solubility of at least 1 gram per liter in deionized water at 20° C.

3. In some embodiments of the granule of paragraph 1 or 2, the core fully dissolves or disperses in less than one minute when 0.5 gram of granules is added to 50 mL of water in a 100 mL beaker stirred at 500 rpm at 25° C.

4. In some embodiments of the granule of any of the preceding paragraphs, the core is sucrose.

5. In some embodiments of the granule of any of the preceding paragraphs, the at least one polyhydroxy compound is a maltodextrin.

6. In some embodiments of the granule of any of the preceding paragraphs, the at least one polyhydroxy compound is sucrose or trehalose.

7. In some embodiments of the granule of any of the preceding paragraphs, at least one phosphate compound is a potassium salt of phosphoric acid.

8. In some embodiments of the granule of any of the preceding paragraphs, the core comprises at least 25% w/w of the granule.

9. In some embodiments, the granule of any of the preceding paragraphs comprises an additional fast-dissolving coating or coatings over or under the protectant matrix.

In some embodiments of the granule of any of the preceeding paragraphs, the granule is a fluidized bed spray-coated granule.

11. In some embodiments of the granule of any of the preceeding paragraphs, the at least one bioactive agent is a microorganism.

12. In some embodiments of the granule of paragraph 11, the microorganism is one or more belonging to to any of the genera selected from the group consising of Bacillus, Paenibacillus, Lactobacillus, Brevibacillus, Escherichia, Gluconobacter, Gluconacetobacter, Acetobacter, Streptococcus, Methylobacterium, Pantoea, Pseudomonas, Sphingomonas, Curtobacterium, Knoellia, Massilla, Pedobacter, Skermanella, Clostridia, Klebsiella, Spirillum, Streptomyces, Coniothyrium, Clonostachys, or Achromobacter, Saccharomyces, Hanseniaspora, Trichoderma, Aspergillus, Aureobasidium, Ulocladium, Muscodor, Metarhizium, Beauveria, Paecilomyces, Isaria, and Lecanicillium.

13. In some embodiments of the granule of paragraph 11, the microorganism is Gluconobacter cerinus and/or Hanseniaspora uvarum.

14. In some embodiments of the granule of any of the preceeding paragraphs, the at least one bioactive agent is an enzyme.

In some embodiments of the granule of paragraph 14, the enzyme is one or more belonging to any of the classes selection from the group consisting of protease, amylase, cellulase, lipase, mannanase, phytase, fucosidase, oxidase, peroxidase, reductase, transferase and transglutaminase.

These and other aspects and embodiments of present modified cells and methods will be apparent from the description, including any accompanying Drawings or Figures.

### BRIEF DESCRIPTION OF THE DRAWINGS

FIG. 1 illustrates the structure and composition of readily dispersible shelf stable bioactive granules of the invention. The granules include a fast-dissolving core surrounded by a coating containing a bioactive agent distributed within a protectant matrix.

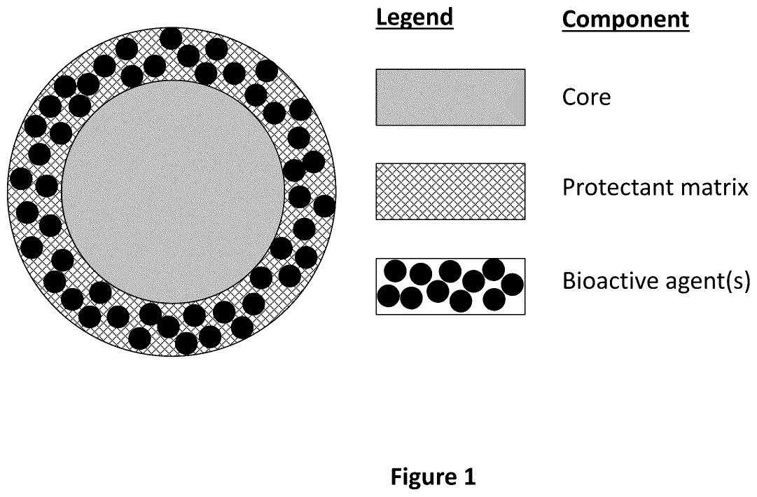

FIG. 2 illustrates the structure and composition of readily dispersible shelf stable bioactive granules with an optional additional coating.

### DETAILED DESCRIPTION

### I. Definitions and Abbreviations

Prior to describing the present compositions and methods in detail, the following terms are defined for clarity. Terms not defined should be accorded their ordinary meanings as used in the relevant art.

As used herein, a “core” is a particle to which a coating can be applied in a fluidized bed spray-coater or other suitable coating apparatus.

As used herein, to “coat” or to “apply a coating” is to spray or otherwise contact a liquid feed suspension so as to evaporate water and other volatile components so as to deposit an integral layer of dried or congealed solids onto a surface or substrate.

As used herein, a “coating” is a layer resulting from the forgoing process.

As used herein, a “protectant mixture” is an aqueous solution comprising one or more polyhydroxy compounds and one or more phosphate compounds, for the purpose of stabilizing or more bioactive agents in the coating layer of a granule.

As used herein, a “matrix” is the dry material in a coating, in which a bioactive agent is distributed.

As used herein, a “protectant matrix” is a matrix resulting from drying a protectant mixture in a coating.

As used herein, a “suspension” is an aqueous solution and/or dispersion comprising dissolved and/or undissolved solids.

As used herein, “suspension solids” or simply “solids” refers to the gravimetric amount (g) or percentage (% w/w) of all soluble and insoluble solids in an aqueous suspension as determined by infrared oven or microwave moisture balance operated without overheating, as per ASTM Standard Test Method for Determination of Total Solids in Biomass, Method E1756-01, or a comparable method. This also corresponds to the solids mass that would be deposited on a fluid bed coated granule, as a percentage of the original mass of the feed suspension, after coating and drying.

As used herein, “moisture content” or “water content” refers to the gravimetric amount (g) or percentage (% w/w) of water in solid material as determined by infrared oven or microwave moisture balance operated without overheating, as per ASTM Standard Test Method for Determination of Total Solids in Biomass, Method E1756-01, or a comparable method.

As used herein, a “bioactive agent” or “bioactive” is microorganism or enzyme with biological activity, such as the ability to catalyze biochemical reactions, produce biologically active metabolic products, inhibit, kill or otherwise control pathogens, facilitate nutrient utilization, neutralize toxins, or otherwise interact to positively influence the heath or viability of living plants, animals or microorganisms.

As used herein, a “microorganism” or “microbe” is any bacteria, fungi or yeast.

As used herein, an “enzyme” is a catalytically active protein.

As used herein, “bioactive fermentation suspension” or simply “fermentation suspension” is a fermentation broth or material derived from a fermentation broth comprising one or more bioactive agents together with any soluble or insoluble media components, metabolites or other impurities derived from the fermentation broth.

As used herein, “bioactive fermentation solids” or simply “fermentation solids” are the suspension solids in a bioactive fermentation broth, either before or after the evaporation of water in a coating and drying process

As used herein, a “feed suspension” is a suspension or solution to be coated onto a core, comprising a combination of bioactive fermentation suspension and a protectant mixture.

As used herein, a “water-soluble” compound is a compound with a solubility of at least 1 gram per liter in deionized water at 20° C.

As used herein, to “fully dissolve” is to leave less than 1% of original solids as residue after pouring an aqueous suspension of the solids through a sieve with a 210-μm mesh (i.e., No. 70).

As used herein, a “fast-dissolving” compound or composition is a compound or composition that fully dissolves or disperses in less than one minute when 0.5 g of compound is added to 50 ml of water in a 100 ml beaker stirred at 50 rpm at 25° C.

As used herein, “particle size” refers to the median diameter of a population of particles, as measured by sieve analysis, or a correlated technique such as laser light scattering or image analysis.

As used herein, “Heubach dust” refers to a dust generated by the Heubach Dust Meter Type III (Heubach DUSTMETER GmbH, Salzburg, Austria). The Heubach test subjects particles to defined crushing and fluidization forces by using rotating paddles to roll steel balls through a bed of granules contained within a cylindrical chamber and simultaneously percolating a stream of air through the bed to strip off any dust that is generated. The generated dust is drawn by vacuum through a tube and deposited onto a filter pad outside the Heubach chamber. The weight or active component of the dust collected is referred to as Heubach dust.

As used herein, the singular articles “a,” “an” and “the” encompass the plural referents unless the context clearly dictates otherwise. All references cited herein are hereby incorporated by reference in their entirety. The following abbreviations/acronyms have the following meanings unless otherwise specified:
aw water activity
° C. degrees centigrade
cfm cubic feet per minute
CFU colony forming units
D₅₀ weight-averaged median diameter
F total solids in feed solution (both soluble and insoluble)
g gram
gpm grams per minute
hr hour
L liter
mg milligram
min minute
mL milliliter
mm millimeter
mM millimolar
psig pounds per square inch, gauge
rpm revolutions per minute
sec second
T_{g} glass transition temperature
% w/w percent weight/weight, also designated simply by % except where defined otherwise
Y spray yield, also called coating efficiency
μm micrometer

### II. Overview of Compositions and Methods for Stabilization of Microorganisms

Previous patents by DePablo el al. (U.S. Pat. Nos. 6,653,062 and 6,919,172) describe the use of a protectant mixture comprising at least one polyhydroxy compound and phosphate ions in certain molar ratios to preserve a wide range of biological materials, such as enzymes, blood cells, or microorganisms, either within an aqueous medium or in a solid form. The polyhydroxy compound can be, for example, a monosaccharide or a monosaccharide. The phosphate ions can be introduced, for example, in the form of simple inorganic phosphate salts such as sodium or potassium phosphate. The patent teaches that if a solid form is desired, the protectant mixture can be dried by a multiplicity of processes, such as freeze-drying, ambient air-drying, vacuum drying, spray-drying, and freezing, and that the resulting compositions are stable for extended periods of time at ambient or super-ambient temperatures and/or relative humidity. The patent exemplifies the use of the aqueous solutions protectant mixtures combined with bacterial cultures, such as Lactobacillus acidophilus and then subjected to freezing, freeze-drying or vacuum drying, and stored in sealed vials at 37° C. or room temperature. Dried formulations that included trehalose or sucrose in combination with phosphate salts showed improved stability over cells alone.

DePablo et al. hypothesize that phosphate ions form a three-dimensional “supermolecular structure” with polyhydroxy compounds via interactions with their hydroxyl groups. The resulting protectant mixture has a higher glass transition temperature (T_{g}) than the corresponding mixture sans the phosphate ions, resulting in a stabilizing structure and reduced mobility of water and degradation mechanisms that involve free water. Publications by Ohtake et. al. [3, 4] further substantiate these claims, demonstrating that sucrose-phosphate and sucrose-trehalose mixture at molar ratios of 0,5:1 or 1:1 show an increase in T_{g} of up to 40° C. over a pH range of 4-9, compared to that of the pure sugars, even at temperatures up to 37° C. and relative humidity up to 40% RH, with residual moisture levels up to 10% w/w. These protectant mixtures furthermore stabilize the lipid bilayer membranes on cells.

The present inventors wished to determine whether a composition like the protectant mixture described by DePablo et al. could stabilize beneficial microbes during extended storage at ambient temperatures and humidity, even outside sealed containers. Microbial suspensions were combined with mixtures of polyhydroxy compounds and phosphate salts, and dried by a spray-granulation process, alternatively known as spouted bed granulation.

In spray-granulation, a feed solution or suspension is continuously sprayed into a fluidized bed fed with heated air, generating primary spray dried particles of fine particle size. The initial spray-dried particles subsequently serve as nuclei upon which additional feed is sprayed, deposited, and layered, to gradually build up and form dry granules. The final size of the resulting granules can be controlled by adjusting process conditions and by continuously withdrawing a sieved fraction of granules that fall within the desired size cutoff range between two sieves. Undersized particles remain within the bed and oversized particles can be separated, milled and re-introduced into the bed. Because nothing besides the feed material is introduced into the spray granulator, the resulting granules are of homogenous composition, consisting solely of particles composed of the dried mixture of protectant compounds and microbial suspension.

As detailed in the Examples, the inventors have found that the spray granulation process can produce well-formed dry granules comprising a microbial suspension distributed within a protectant matrix. The protectant matrix used in the examples is composed exclusively of water-soluble polyhydroxy compounds and water-soluble source of phosphate ions, namely sucrose, maltodextrin, monopotassium phosphate and dipotassium phosphate. The granules formed by this process have a particle size in the range of about 250-2000 microns and are free flowing and relatively non-dusty. These spray-granulated granules exhibit an excellent shelf life over more than a month when stored under ambient conditions.

However, despite the solubility of the individual components, the dry microbial granules produced using the aforementioned protectant mixture and microbial suspension nevertheless do not readily dissolve or disperse in water or aqueous solution. Even with agitation, it takes more than a minute, typically even more than 3 minutes, to fully dissolve or disperse the spray-granulated microbial granules in water, even with agitation. This dissolution rate is inadequate for some applications.

The present invention provides a granule structure and composition that simultaneously achieves the objectives of rapid dissolution or dispersibility, low residue, extended shelf life at ambient conditions, low dust, and good flowability. This is particularly remarkable because there is a tension between the objectives of rapid dissolution in water and yet at the same time extended shelf life in the presence of ambient humidity and temperature. The inventors have surprisingly found that these simultaneous objectives can be achieved by means of a layered granule, comprising a core surrounded by a coating layer comprising microorganisms within a protectant matrix, wherein the protectant matrix comprises at least one polyhydroxy compound and at least one phosphate compound. The core can be any material that is water-soluble and fast-dissolving, for example sucrose or sodium sulfate. The polyhydroxy compounds can be any polysaccharide, disaccharide or monosaccharide that is water-soluble and fast-dissolving. For example, the polyhydroxy compounds can be maltodextrin, sucrose or trehalose. The water-soluble phosphate can be any inorganic or organic phosphate or phosphate salt that is water-soluble and fast-dissolving, for example a potassium salt of phosphoric acid, such as monopotassium phosphate or dipotassium phosphate.

The granule of can be produced by any suitable coating process for applying a coating layer of the microorganisms in a water-soluble matrix. A preferred coating process for producing the granule is fluidized bed spray-coating.

Without wishing to be bound by theory, it has been observed by the present inventors that the mixture of phosphate ions and polyhydroxy compounds results in aqueous solution results in an increase in viscosity of the solution, which is consistent with formation of the “super-molecular structure” that DePablo et al. deduced based upon its elevated glass transition temperature. This super-molecular structure persists when the protectant mixture is dried into a particle of homogeneous composition that includes the bioactive suspension and could account for the tendency to dissolve more slowly when rehydrated.

The new element provided by the coated structure of the invention is the incorporation of a water-soluble and fast-dissolving core material such as sucrose. Upon addition of the readily dispersible granule into bulk water, the fast-dissolving core provides a strong driving force to draw water through the coating layer comprising protectant matrix, thereby accelerating its dispersal and dissolution. Without the fast-dissolving core, the protectant mixture by itself takes significantly longer to hydrate and fully dissolve, since the protectant mixture in the matrix is water-soluble but slower to spontaneously hydrate than the fast-dissolving core. Furthermore, the layered structure of the granule distributes the protectant matrix to a thinner coating layer at the particle surface, making it more readily accessible and faster to dissolve than for the corresponding spray granule with a unitary structure with most of the protectant matrix buried below the particle surface. To achieve these beneficial rapid-dispersion and solubilization effects, the core should comprise at least 25% of the mass of the coated granule.

It should be noted that in the referenced disclosures of DePablo et al. there are no observations reported regarding dissolution or dispersion kinetics of the solid materials produced using the protectant mixture, nor any suggestion of using a coated particle structure or any other inhomogeneous structure to accelerate or otherwise modify the kinetics of dissolution and dispersion thereof. Accordingly, such data are provided, herein, for comparison.

### III. Materials for Use in Readily Dispersible Shelf Stable Granules

Suitable cores of the invention are water-soluble and fast-dissolving. Particles composed of soluble or insoluble inorganic salts and/or sugars and/or small organic molecules may be used as cores. Suitable water-soluble ingredients for incorporation into cores include: sugars such as sucrose, trehalose, glucose, fructose or lactose; sugar alcohols such as sorbitol, or mannitol; polysaccharides such as maltodextrin or soluble starch; inorganic salts such as ammonium sulfate, sodium sulfate, magnesium sulfate, zinc sulfate, sodium chloride or potassium chloride; organic acids such as citric acid, succinic acid or lactic acid; and osmolytes such as urea and betaine, and the like.

Polyhydroxy compounds used in protectant mixtures include water-soluble, non-polysaccharides, disaccharides and monosaccharides. Polysaccharides include maltodextrin and soluble starch, or starch hydrolysates, derived from corn, sorghum, arrowroot, rice, wheat, rye, barley, oat, potato, yam, tapioca, cassava, and sago. Mono- and disaccharides include sugars such sucrose, trehalose and lactose; or sugar alcohols such as sorbitol or mannitol.

Phosphate compounds used in protectant mixtures include water-soluble, fast-dissolving inorganic and organic phosphate compounds such as sodium and potassium salts of phosphoric acid such as monosodium phosphate and disodium phosphate.

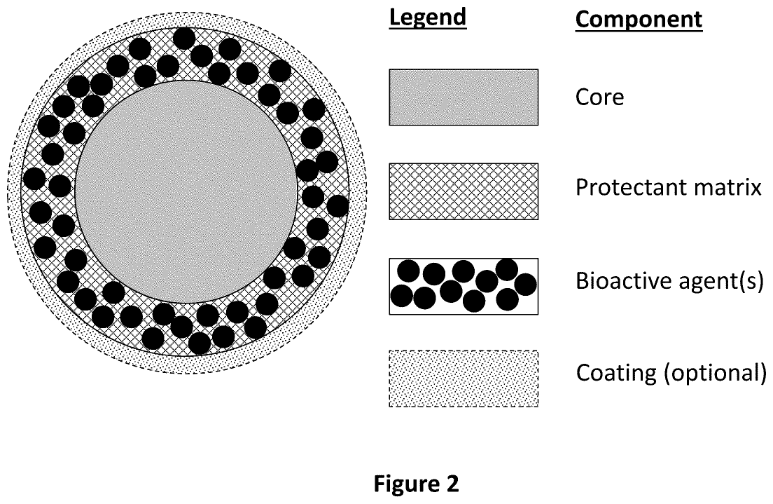

The granule can comprise an additional coating or coatings over or under the protectant matrix, as shown in FIG. 2. Optional coatings must comprise fast-dissolving materials.

The granule comprises at least one bioactive agent. Bioactive agents are living organisms, or compounds derived from living organisms, exhibiting biological activity, such as the ability to catalyze biochemical reactions, produce biologically active metabolic products, inhibit, kill or otherwise control pathogens, facilitate nutrient utilization, neutralize toxins, or otherwise interact to positively influence the heath or viability of living plants, animals or microorganisms. Bioactive agents include microorganisms and enzymes.

### IV. Bioactive Agents Suitable for Delivery in Readily Dispersible Shelf Stable Granules

Microorganisms include any bacteria, yeast, or fungi. Suitable bacteria include gram-positive or gram-negative bacteria, including but not limited to bacteria of the genera Bacillus, Paenibacillus, Lactobacillus, Brevibacillus, Escherichia, Gluconobacter, Gluconacetobacter, Acetobacter, Streptococcus, Methylobacterium, Pantoea, Pseudomonas, Sphingomonas, Curtobacterium, Knoellia, Massilla, Pedobacter, Skermanella, Clostridia, Klebsiella, Spirillum, Streptomyces, Coniothyrium, Clonostachys, and/or Achromobacter. Suitable yeast include, but are not limited to, yeast of the genera Saccharomyces, Hanseniaspora. Suitable fungi include, but are not limited to, fungi of the genera Trichoderma, Aspergillus, Aureobasidium, Ulocladium, Muscodor, Metarhizium, Beauveria, Paecilomyces, Isaria, and/or Lecanicillium.

Enzymes suitable for delivery in readily dispersible shelf stable granules include, but are not limited to, any protease, amylase, cellulase, lipase, mannanase, phytase, fucosidase, oxidase, peroxidase, reductase, transferase or transglutaminase. Enzymes can be used in applications such as laundry and dish detergents, textile processing, agriculture and crop protection, animal health and nutrition, or human health and nutrition.

### V. Coating Processes and Equipment Suitable for Producing Readily Dispersible Shelf Stable Granules

In general, any coating process or equipment can be used to coat the feed suspension onto the cores. The coating process can be a fluidized bed spray-coating process, started by introducing and suspending cores in a fluidized air stream and spray an atomized coating feed solution or suspension comprising a benefit agent and cross-linkable polymer so as to contact and deposit successive layers of dried material onto the cores upon evaporation of the water or other solvent from the coating feed, thereby building up a continuous coating or “shell.” The cores may be inert particles devoid of a benefit agent or may alternatively comprise one or more benefit agents.

Fluidized bed spray-coating processes can be carried in using different fluidized bed spray-coater coater equipment configurations, including top-spray fluidized bed, bottom-spray (also known as Wurster) fluidized bed, spray-agglomeration, in which the coated cores are further build up by recirculation and bridging to form larger clusters or agglomerates, whereby the coating solution dries to create particles that comprise multiple cores bridged by dried coating solution. These fluidized bed processes can be operated in batch mode wherein all the cores are introduced into the fluidized bed at one time, or in continuous mode, wherein new core material is added and withdrawal continuously or periodically.

The coating process can be carried out by mechanical coater, via a mixing or agitation process, in which core particles are coated by spraying or flowing a coating solution onto cores undergoing vigorous agitation by various means, such as tumbling or rotation on a friction place, to enable deposition, spreading and drying of the coating. Mechanical coaters include mixing and agitation processes that can be carried out using different equipment configurations, such as drum granulators, mixer-granulators, ribbon blenders, V-blenders, twin-shell blenders, conical blenders, Nauta mixers and other conical screw mixers, high-shear granulators, spheronizers, roto granulators, and the like.

### EXAMPLES

The following examples are intended to illustrate but not limit the present compositions and methods.

### Example 1. Production of Granules Containing Bioactives

A batch of granules, designated “Granule(s) A,” was produced by fluidized-bed batch spray coating. Granule A consisted of fermentation solids of Gluconobacter cerinus and Hanseniaspora uvarum, as well as the excipients sucrose, maltodextrin, KH₂PO₄, K₂HPO₄, and Ca₃(PO₄)₂.

To prepare the granules, a co-fermentation of G. cerinus and H. uvarum was performed, and approximately 30 L of the resulting fermentation broth was concentrated by a factor of 2.8 using a centrifuge, increasing the dry solids percentage from 6% to 17%. A Vector VFC-LAB1 fluidized-bed spray coater (Freund-Vector, Marion, Iowa, USA) was charged with 650 g of granular sucrose cores with a median diameter (D₅₀) of 410 μm. The microbe-containing concentrate was mixed with sterilized solutions of sucrose, maltodextrin, KH₂PO₄, and K₂HPO₄. The maltodextrin used in solution preparation was Glucidex Premium 12 maltodextrin (Roquette, Lestrem, France).

The resulting mixture was spray-coated onto the sucrose-containing cores at a spray rate of 10-15 g/min. During the spray-coating process, the temperature in the fluidized bed was held between 50-55° C., and the relative humidity was held between 20-30%. The fluidization air was held between 70-80 cubic feet per minute (cfm) and the atomization air pressure was held between 35-40 psig. The granules were removed from the spray-coater into a plastic container and shaken together with 1 gram of Ca₃(PO₄)₂ as an anti-caking agent.

Finished Granule A consisted of approximately 50% sucrose cores, 18% fermentation solids, 6% sucrose in the spray-coating mixture, 13% maltodextrin, 5% KH₂PO₄, 8% K₂HPO₄, and less than 1% Ca₃(PO₄)₂. Approximately 1,330 g of Granule A was generated by this process. The D₅₀ for Granule A was 570 μm. The water activity of Granule A immediately after production was 0.19. Viability of G. cerinus above 10¹⁰ CFU/g and of H. uvarum above 10⁹ CFU/g in Granule A was confirmed by plating.

A second batch of granules, designated “Granule(s) B,” was produced by fluidized-bed batch spray-coating. Granule B consisted of fermentation solids of Lactobacillus reuteri, as well as the excipients sucrose, maltodextrin, KH₂PO₄, K₂HPO₄, and Na₂SO₄.

To prepare the granules, a fermentation of L. reuteri was carried out. To the fermentation broth, sucrose, KH₂PO₄, K₂HPO₄, were added, and the mixture was frozen into pellets by dropping into liquid nitrogen. A Vector VFC-LAB1 fluidized-bed spray coater was charged with 650 g of granular sucrose cores with a D₅₀ of 410 μm. The microbe-containing pellets were thawed to 4° C. and mixed with a sterilized solution of maltodextrin. The maltodextrin used in solution preparation was Glucidex Premium 12 maltodextrin (Roquette, Lestrem, France). The resulting mixture was spray-coated onto the sucrose cores at a spray rate of 10-15 g/min. During the spray-coating process, the temperature in the fluidized bed was held between 50-55° C., and the relative humidity was held between 20-30%. The fluidization air was held between 70-75 cfm and the atomization air pressure was held between 35-40 psig. The granules were subsequently coated with an additional layer of Na₂SO₄, sprayed from a 25% solution with temperatures in the fluidized bed held between 48-52° C., and relative humidity in the fluidized bed held between 15-35%. During spray-coating of the Na₂SO₄ layer, the fluidization air was held between 70-75 cfm, and the atomization air was held between 32-37 psig.

Finished Granule B consisted of approximately 35% sucrose cores, 12% fermentation solids, 4% sucrose, 7% maltodextrin, 4% KH₂PO₄, 7% K₂HPO₄in the spray-coated protectant matrix coating, and a further 31% NaSO₄ coating over the protectant matrix coating. Approximately 2,100 g of Granule B was generated by this process. The D₅₀ for Granule B was 610 μm. The presence of viable L. reuteri in Granule B was confirmed by plating.

A third batch of granules, designated “Granule(s) C,” was produced by fluidized-bed batch spray coating as described in WO2020/086821. Granule C consisted of fermentation solids of Metarhizium anisopliae and the excipients Na₂SO₄, polyvinyl alcohol (PVA), trehalose and talc.

A fermentation of M anisopliae was carried out. A Vector VFC-LAB1 fluidized-bed spray coater was charged with 930 g of Na₂SO₄ cores with a D₅₀ of 220 μm. Broth from the M anisopliae fermentation was mixed with a sterilized suspension of PVA, trehalose, and talc and used spray-coated onto the Na₂SO₄ cores. During the spray-coating process, the temperature in the fluidized bed was held between 35-40° C., and the relative humidity was held between 40-50%. The material was sprayed at a rate of 10-15 g/min, the fluidization air was held between cfm and the atomization air pressure was held between 30-35 psig. The granules were subsequently coated with an additional layer of Na₂SO₄, sprayed from a 25% solution at a rate of 18-22 g/min, with temperatures in the fluidized bed held between 35-40° C., and relative humidity held between 15-35%. The fluidization air was held between 55-60 cfm and the atomization air pressure was held between 30-35 psig. The final quantity of Granule C produced was approximately 1,680 g. The D₅₀ for Granule C was 420 μm.

Finished Granule C contained approximately 47% Na₂SO₄ core, 8% M anisopliae fermentation solids, 12% talc, 8% trehalose, 4% PVA, and 21% Na₂SO₄ sealing layer. Viability of M anisopliae in Granule C above 10⁵ CFU/g was confirmed via plating.

A further batch of granules, designated “Granule(s) D,” were produced by fluidized-bed continuous spray granulation. Granule D consists of fermentation solids of G. cerinus and H. uvarum, as well as the excipients sucrose, maltodextrin, KH₂PO₄, K₂HPO₄, and Ca₃(PO₄)₂.

To prepare the granules, a co-fermentation of G. cerinus and H. uvarum was performed, and approximately 20 L of the resulting fermentation broth was concentrated by a factor of 2.5 using a centrifuge, increasing the dry solids percentage from 6% to 15%. A Glatt ProCell Lab System 5 unit (Glatt GmbH, Weimar, Germany) was charged with 100 g maltodextrin. The microbe-containing concentrate prepared on the centrifuge was mixed with sterilized solutions of sucrose, maltodextrin, KH₂PO₄, and K₂HPO₄. The maltodextrin used in solution preparation was Glucidex Premium 12 maltodextrin (Roquette, Lestrem, France). The resulting mixture was spray-granulated in a continuous fluidized-bed process using the ProCell Lab System 5 using a spray rate between 15-22 g/min. During the spray-granulation process, the temperature in the fluidized bed was held between 50-55° C., and the relative humidity was held between 10-25%. The fluidization air was held between 140-150 m³/h and the atomization air pressure was held between 4.2-4.7 bar. The granules were removed from the spray-granulation unit into a plastic container and shaken together with 1 g Ca₃(PO₄)₂ as an anti-caking agent.

Finished Granule D contained approximately 33% fermentation solids, 17% sucrose, 22% maltodextrin, 11% KH₂PO₄, 17% K2HPO₄, and less than 1% Ca₃(PO₄)2. Approximately 500 g of Granule D was generated by this process. The D₅₀ for Granule D was 1,600 μm. Viability of G. cerinus above 10¹⁰ CFU/g and of H. uvarum above 10⁹ CFU/g in Granule A was confirmed by plating.

A further batch of granules, designated “Granule(s) E,” was produced by fluidized-bed continuous spray granulation. Granule E consisted of fermentation solids of G. cerinus and H. uvarum, as well as the excipients PVA, talc, and trehalose.

To prepare the granules, a co-fermentation of G. cerinus and H. uvarum was performed, and approximately 40 L of the resulting fermentation broth was concentrated by a factor of 2.6 using a centrifuge, increasing the dry solids percentage from 5% to 13%. A Glatt ProCell Lab System 5 unit was charged with 100 g maltodextrin. The fermentation broth was mixed with a sterilized suspension of PVA, trehalose, and talc and spray-granulated in a continuous fluidized-bed process using the ProCell LabSystem 5 at a spray rate between 18-25 g/min. During the spray-granulation process, the temperature in the fluidized bed was held between 45-50° C. and the relative humidity between 20-30%. The fluidization air was held between 140-150 m³/h and the atomization air pressure was held between 4.2-4.7 bar.

Finished Granule E consisted of approximately 11% fermentation solids, 25% PVA, 51% talc, and 13% trehalose. Approximately 600 g of Granule E was generated by this process. The D₅₀ for Granule E was 760 μm. Viability of G. cerinus above 10⁸ CFU/g and of H. uvarum above 10⁶ CFU/g in Granule E was confirmed by plating.

Further granules, designated “Granule(s) F,” were produced by freeze-drying according to methods described in the patents of DePablo et al. (U.S. Pat. Nos. 6,653,062 and 6,919,172). The granules consisted of fermentation solids of a Bifidobacterium lactis, as well as the excipients sucrose, KH₂PO₄, and K₂HPO₄. The mixture was freeze-dried to produce granules with a particle size in the range of 1-5 mm. The viability of B. lactis in Granule F was confirmed to be above 10¹¹ CFU/g by plating.

Further granules, designated “Granule(s) G”, were obtained. Granule G is a sample of the commercial solid product BioWorks Rootshield Plus WP (BioWorks, Inc, Victor, NY), containing the microbes Trichoderma harzianum T-22 and Trichoderma virens G-41, and is intended for use in agricultural applications, including applications that require dispersal of the granules in water, including irrigation or spraying.

Further granules, designated “Granule(s) H,” were produced by fluidized-bed batch spray coating. Granule H consists of fermentation solids of a genetically modified variant of T. reesei overexpressing phytase, as well as the excipients Na₂SO₄, polyvinyl alcohol (PVA) and talc.

To prepare the granules, a Vector VFC-LAB1 fluidized-bed spray coater was charged with 600 g of Na₂SO₄ cores with a D₅₀ of 220 μm. A layer of fermentation solids mixed with PVA was spray-coated onto the cores at a bed temperature between 42-45 ° C., with a spray rate between 8-10 gpm, fluidization air between 50-52 cfm, and atomization air between 30-35 psig. A layer of PVA mixed with talc at a total dry solids ratio of 18% was subsequently spray-coated onto the cores at a bed temperature between 47-52 ° C., with a spray rate between 8-12 gpm, fluidization air between 51-53 cfm, and atomization air between 37-42 psig. A layer of Na₂SO₄ was subsequently spray-coated onto the cores from a 25% solution at a bed temperature between 36-55 ° C., with a spray rate between 25-30 gpm, fluidization air between 53-54 cfm, and atomization air between 37-42 psig. A layer of PVA mixed with talc at a total dry solids ratio of 18% was subsequently spray-coated onto the cores at a bed temperature between 47-52 ° C., with a spray rate between 8-12 gpm, fluidization air between 51-53 cfm, and atomization air between 37-42 psig.

The finished Granule H consisted of approximately 8% fermentation solids, 5% PVA, 7% talc, and 80% Na₂SO₄. Approximately 2200 g of Granule H was generated by this process. The Dso for Granule H was 240 μm. The resulting phytase granules exhibited phytase activity above 10⁴ phytase transfer units per gram (FTU/g) as measured by the standard malachite green phosphate assay for phytase.

### Example 2. Dissolution Rate of a Readily Dispersible Granule in Comparison to an Alternative Stabilizing Chemistry

Granules A, B, and C were subjected to a dissolution test. In a single trial of the dissolution test, 50 mL of tap water were dispensed into a beaker. The interior diameter of the bottom of the beaker was measured to be 42 mm. An oblong, plastic-coated magnetic stir-bar, with a length measured at 25 mm, was used to agitate the samples at a stirring rate of 500 rotations per minute (rpm). A 500-mg aliquot of the granules was added to the beaker all at once, and a stopwatch was started. After preselected periods of time, magnetic stirring was stopped, and material was passed through a sieve with 210-μm mesh (i.e., No. 70). If no solid material was retained on the sieve, the granule was recorded to be “dissolved” at that time point. If any solid material was retained on the sieve, the granule was recorded to be “undissolved” at that time point. The experiment was conducted at ambient conditions, in a lab with a temperature maintained between 22-24° C. and humidity maintained between 50-60% during the course of the experiment.

The foregoing dissolution test was repeated five times for each sample at a variety of time points between 1 and 5 minutes. The number of dissolved results was recorded for each sample at each time point. A sample with more dissolved results at earlier time points was considered to be more soluble than a sample with fewer dissolved results. The results of the dissolution experiment for Granules A, B and C are summarized in Table 1.

TABLE 1
Dissolution results for Granules A, B and C

| | | Number of Dissolved Results at | | | |
|---|---|---|---|---|---|
| | Matrix | Each Time Point, n = 5 trials | | | |
| Granule | Chemistry | 1 min | 2 min | 3 min | 5 min |
| Granule A | Sucrose- | 4 | 5 | 5 | 5 |
| | phosphate- | | | | |
| | maltodextrin | | | | |
| Granule B | Sucrose- | 5 | 5 | 5 | 5 |
| | phosphate- | | | | |
| | maltodextrin | | | | |
| | with Na₂SO₄ | | | | |
| | coating | | | | |
| Granule C | PVA-talc- | 0 | 0 | 3 | 5 |
| | trehalose | | | | |

The results in Table 1 show that Granules A and Granule B are significantly faster dissolving than Granule C. Granules A and B dissolve completely within approximately 1 minute of stirring under the described conditions, whereas Granule C requires mixing for 3 minutes or more to achieve complete dissolution. This result is despite the larger particle size of both Granule A (570 μm) and Granule B (610 μm) compared to Granule C (420 μm). This result is also despite the presence of the additional external protective Na₂SO₄ coating of Granule B. The sucrose-phosphate-maltodextrin formulation is clearly more rapidly soluble than the PVA-talc-trehalose formulation. The sucrose-phosphate-maltodextrin coated granule formulation is, therefore, more suitable for a range of applications, including agriculture or animal nutrition, where mixing capabilities in the site of commercial application may be limited, and rapid dissolution is essential.

### Example 3. Dissolution Rate of a Readily Dispersible Granule in Comparison to Similar Excipient Chemistries Produced Using Alternative Drying Methods

Granules A, B, D, and F were each tested using the dissolution protocol described in Example 2. All four granules contain microbial solids, sucrose, and K₂HPO₄, and KH₂PO₄. The results are summarized in Table 3.

TABLE 3
Dissolution results for microbe-containing Granules A, B, D, and F

| | | Number of Dissolved Results | | | | | |
|---|---|---|---|---|---|---|---|
| | Production | at Each Time Point, n = 5 trials | | | | | |
| Granule | Method | 1 min | 2 min | 3 min | 5 min | 7 min | 10 min |
| Granule A | Spray- | 4 | 5 | 5 | 5 | 5 | 5 |
| | coating onto | | | | | | |
| | sucrose cores | | | | | | |
| Granule B | Spray- | 5 | 5 | 5 | 5 | 5 | 5 |
| | coating onto | | | | | | |
| | sucrose cores, | | | | | | |
| | followed by | | | | | | |
| | Na₂SO₄ | | | | | | |
| | coating | | | | | | |
| Granule D | Spray- | 0 | 3 | 5 | 5 | 5 | 5 |
| | granulation | | | | | | |
| | (no cores) | | | | | | |
| Granule F | Freeze- | 0 | 0 | 1 | 0 | 1 | 4 |
| | drying (no | | | | | | |
| | cores) | | | | | | |

The results in Table 3 show that Granules A and B are more rapidly soluble than Granule D. Granules A and B typically dissolved completely within 1 minute of stirring under the conditions described, whereas Granule D required 2-3 minutes of stirring to achieve complete dissolution. Clearly, excipient chemistry alone does not determine the dissolution rate of the granules. The production method and granule structure also determines the rate at which the granules dissolve. In fact, Granule B dissolves at least as quickly as Granule A, despite the addition of a protective Na₂SO₄ coating. In applications such as agriculture or animal nutrition, where mixing capabilities at the site of application may be limited, rapid dissolution is essential, and a readily dispersible granule such as Granule A or B is strongly preferred over Granule D.

The results in Table 3 additionally show that Granules A and B are more rapidly soluble than Granule F. Despite the similar chemistries, Granules A and B are typically dissolved completely within 1 minute of stirring at the conditions described, whereas Granule F requires 10 minutes to achieve complete dissolution. A large mass fraction of Granule F, at least 90%, appeared to dissolve rapidly in the dissolution test. However, in most of the dissolution tests, there remained between one and five larger, agglomerated structures that accumulated on the 210-μm sieve. Only after 10 minutes of stirring did these larger structures begin to disperse, leaving the sieve free of solids when the dissolution test was performed.

Practical applications of bioactive-containing granules such as Granules A and F may involve dispensing much larger quantities than 500 mg as used in these tests. The agglomerates observed in dissolution of Granule F could accumulate on the bottom of a mixing tank and cause dosing issues or require additional cleaning. In an application such as a spray for agriculture, the agglomerates could accumulate on the mesh filter of a spray tank and cause clogging of lines or plugging of spray nozzles in delivery. In other applications, such as human or animal consumption of the granules, or in-furrow application for agriculture, the presence of the agglomerates may not present an issue. However, in many applications, the rapid and complete dissolution of Granules A and B is very much preferred.

### Example 4. Dustiness of a Readily Dispersible Granule in Comparison to Alternative Production Methods

Granules A, B, D, F, and G were assayed for dustiness using a Heubach Dustmeter Type III (Heubach, Fairless Hills, PA). Samples were loaded volumetrically to 16.25 ml fill volume, based on bulk density into the grinding chamber. The program was set to run at 45 rpm for 20 minutes. Airflow was 20 L/min. Accumulated dust was weighed and recorded as a percentage of the weight of the sample. The dust levels measured for Granules A, B, D, F, and G are summarized in Table 5.

TABLE 5
Heubach III Dust Results for Granules A, F and G

| | Heubach |
|---|---|
| | III Dust |
| Granule | Level |
| Granule A | 0.7% |
| Granule B | 0.8% |
| Granule D | 1.0% |
| Granule F | 2.8% |
| Granule G | 7.1% |

The results in Table 2 show that Granules A and B are the least dusty of the granules tested, and are significantly less dusty than Granules F and G. A formulation using the readily dispersible granule formulation is less dusty than a formulation using a freeze-drying formulation, which is a common method of preserving the viability of live microbes. The commercially available Granule G, intended for use in agriculture, is also dusty. Of the three granules, Granules A and B, which have the additional benefit of dispersibility described in Example 1, are also the least dusty granules. A low dust level is preferable for dosing, cleaning, and safety.

### Example 5. Microbial Stability of a Readily Dispersible Granule in Comparison to an Alternative Drying Method and an Alternative Chemistry

The viability of G. cerinus and H. uvarum in Granules A, D and E was tested by dilution plating. Each viability test was performed according to the following protocol. A 0.3-g aliquot of each granule was dissolved in 5.7 g water. The resulting suspensions were serially diluted and appropriate dilutions were evenly distributed on the surfaces of Petri dishes containing agar medium (PDA agar for G. cerinus and YPD agar for H. uvarum). The inoculated agar plates were incubated in an incubator at 25° C. for 3 days. During this time, viable microbial structures from the fermentation solids produced colonies on the plate. The colonies were counted and reported as colony-forming units per gram of granules (CFU/g).

To test stability of the microbes in the granules over time, 5-g aliquots of the granules were held in 15-mL tubes in an incubator at 25° C. and 55% relative humidity. The viability of the microbes in the granules was tested at regular intervals according to the protocol above. The results are listed in Tables 6 and 7, below.

TABLE 6
Microbial stability results for G. cerinus microbe-containing Granules A, D and E

| | | | Viability after incubation 25° C. and 55% | | | | |
|---|---|---|---|---|---|---|---|
| | Production | Matrix | RH for specified length of time, CFU/g | | | | |
| Granule | Method | Chemistry | 0 weeks | 1 weeks | 2 weeks | 4 weeks | 6 weeks |
| Granule A | Spray-coating | Sucrose- | 7 × 10¹⁰ | 5 × 10¹⁰ | 7 × 10¹⁰ | 5 × 10¹⁰ | 5 × 10¹⁰ |
| | onto sucrose | phosphate- | | | | | |
| | cores | maltodextrin | | | | | |
| Granule D | Spray- | Sucrose- | 5 × 10¹⁰ | 6 × 10¹⁰ | 6 × 10¹⁰ | 5 × 10¹⁰ | 5 × 10¹⁰ |
| | granulation | phosphate- | | | | | |
| | (no cores) | maltodextrin | | | | | |
| Granule E | Spray- | PVA-talc- | 5 × 10⁸ | 5 × 10⁵ | 3 × 10⁵ | 0 | 0 |
| | granulation | trehalose | | | | | |
| | (no cores) | | | | | | |

TABLE 7
Microbial stability results for H. uvarum microbe-containing Granules A, D, and E

| | | | Viability after incubation 25° C. and 55% | | | | |
|---|---|---|---|---|---|---|---|
| | Production | Matrix | RH for specified length of time, CFU/g | | | | |
| Granule | Method | Chemistry | 0 weeks | 1 weeks | 2 weeks | 4 weeks | 6 weeks |
| Granule A | Spray-coating | Sucrose- | 3 × 10⁹ | 2 × 10⁹ | 3 × 10⁹ | 3 × 10⁹ | 3 × 10⁹ |
| | onto sucrose | phosphate- | | | | | |
| | cores | maltodextrin | | | | | |
| Granule D | Spray- | Sucrose- | 4 × 10⁹ | 5 × 10⁹ | 4 × 10⁹ | 3 × 10⁹ | 3 × 10⁹ |
| | granulation | phosphate- | | | | | |
| | (no cores) | maltodextrin | | | | | |
| Granule E | Spray- | PVA-talc- | 3 × 10⁶ | 1 × 10⁵ | 3 × 10⁵ | 0 | 0 |
| | granulation | trehalose | | | | | |
| | (no cores) | | | | | | |

The results in Tables 6 and 7 show that Granules A and D have superior stability of viable organisms compared to Granule E. The sucrose-phosphate-maltodextrin formulation provides improved stability properties for both microbes in comparison to the PVA-talc-trehalose formulation. Whereas the viability of Granules A and D was mainly retained through 6 weeks, viability of Granule E had been lost by 4 weeks. As described, above, Granule A has the additional benefit of improved dissolution and lower dust in comparison to Granules D and E.

### Example 6. Extending Microbial Stability of a Readily Dispersible Granule with Packaging

Five 20-g aliquots of Granule A were stored for 10 days in each of five configurations: (1) at 30° C., 75% relative humidity in an open container; (2) at 30° C., 75% relative humidity in a sealed, soluble PVA bag; (3) at 30° C., 75% relative humidity in a sealed, soluble PVA bag that is sealed inside a mylar bag; (4) at 30° C., 75% relative humidity in a sealed, soluble PVA bag that is sealed inside a mylar bag with a silica gel pack (Uline, Pleasant Prairie, WI, USA); (5) at 25° C., 55% relative humidity in a closed tube, the control condition. The viability of the granules was measured after 10 days incubation at these conditions. The viability results are described in Table 8.

TABLE 8
Microbial stability results for H. uvarum and
G. cerinus under various storage conditions

| | | | Viability of listed | |
|---|---|---|---|---|
| | | | microbe after 10 days storage | |
| | Storage | Packaging | at listed condition, CFU/g | |
| Sample | Conditions | Method | G. cerinus | H. uvarum |
| 1 | 30° C., | Open | 0 | 0 |
| | 75% RH | container | | |
| 2 | 30° C., | PVA bag | 0 | 0 |
| | 75% RH | | | |
| 3 | 30° C., | Mylar bag | 3 × 10¹⁰ | 1 × 10⁹ |
| | 75% RH | containing | | |
| | | PVA bag | | |
| 4 | 30° C., | Mylar bag | 4 × 10¹⁰ | 3 × 10⁹ |
| | 75% RH | containing | | |
| | | PVA bag with | | |
| | | silica gel | | |
| 5 | 25° C., | Closed tube | 4 × 10¹⁰ | 2 × 10⁹ |
| | 55% RH | (control) | | |

The results in Tables 7 and 8 show that packaging solutions can be used to preserve the viability of the readily dispersible Granule A at extreme, superambient temperature and humidity conditions that exert additional stresses on the granules. Additionally, when packaged in a mylar bag, the granules retain their superior flowability and solubility properties. Samples 1 and 2 were heavily crystallized, not flowable, and poorly soluble. Samples 3 and 4 were easily flowable and had comparable solubility properties to Sample 5. The packaging solutions allow preservation of the favorable properties of the readily dispersible granule at high, superambient temperature and humidity storage conditions.

### Example 7. Readily Dispersible Granule Containing Enzymes

Granules A and H were tested according to the dissolution protocol described in Example 2. The results of the dissolution test are described in Table 9.

TABLE 9
Dissolution results for microbe-containing Granules A and H

| | | Number of Dissolved Results | | | | |
|---|---|---|---|---|---|---|
| | | at Each Time Point, n = 5 trials | | | | |
| Granule | Formulation | 1 min | 2 min | 3 min | 5 min | 7 min |
| Granule A | Spray-coating onto | 4 | 5 | 5 | 5 | 5 |
| | sucrose cores in sucrose- | | | | | |
| | maltodextrin-phosphate | | | | | |
| | matrix coating | | | | | |
| Granule H | Spray-coating onto | 0 | 2 | 4 | 5 | 5 |
| | Na₂SO₄ cores in PVA- | | | | | |
| | talc-Na₂SO₄ matrix | | | | | |
| | coating | | | | | |

The results in Table 9 show that Granule A is more soluble than Granule H. Whereas Granule A dissolves readily within 1 minute, Granule H requires between 3-5 minutes to dissolve fully. The higher solubility of Granule A would make this formulation more appropriate for a variety of applications.

Aliquots of Granule H were placed in closed containers and stored at 30° C., 65% relative humidity conditions. After 6 weeks of storage at these conditions, the phytase activity was measured using the standard malachite green phosphate assay for phytase, and it was determined that between 80-85% of the enzyme activity was retained. The microbial stability results in Example 7 suggest that the formulation and packaging solutions described would allow production of an enzyme-containing granule with similar stability properties, but improved dispersibility properties.

### REFERENCES

[1] T. Becker, G. Park, A. L. Gaertner, Chapter 15 in J. H. van Ee, 0. Misset, E. Bass, “Enzymes in Detergency”, New York: Marcel Dekker, 1997.

[2] K. Kadam, Chapter 12 in “Granulation Technology for Bioproducts”, Boca Raton: CRC Press, 1991.

[3] S. Ohtake, C. Schebor, S. P. Palecek, J. J. de Pablo, Pharmaceutical Research, 21(9) (2004) 1615-1621.

[4] S. Ohtake, C. Schebor, S. P. Palecek, J. J. de Pablo, Cryobiology 48 (2004) 81-89.

1. A layered granule comprising a core surrounded by a coating layer comprising at least one bioactive agent distributed within a protectant matrix, wherein the protectant matrix comprises:
a. at least one polyhydroxy compound; and
b. at least one phosphate compound;
and wherein the at least one polyhydroxy compound, the at least one phosphate compound, and the core are water soluble and fast-dissolving.

2. The granule of claim 1, wherein the core has a solubility of at least 1 gram per liter in deionized water at 20° C.

3. The granule of claim 2, wherein the core fully dissolves or disperses in less than one minute when 0.5 gram of granules is added to 50 mL of water in a 100 mL beaker stirred at 500 rpm at 25° C.

4. The granule of claim 3, wherein the core is sucrose.

5. The granule of claim 4, wherein the at least one polyhydroxy compound is a maltodextrin.

6. The granule of claim 5, wherein the at least one polyhydroxy compound is sucrose or trehalose.

7. The granule of claim 6, wherein at least one phosphate compound is a potassium salt of phosphoric acid.

8. The granule of claim 7, wherein the core comprises at least 25% w/w of the granule.

9. The granule of claim 8, comprising an additional fast-dissolving coating or coatings over or under the protectant matrix.

10. The granule of claim 9, wherein the granule is a fluidized bed spray-coated granule.

11. The granule of claim 10, wherein the at least one bioactive agent is a microorganism.

12. The granule of claim 11, wherein the microorganism is one or more belonging to to any of the genera selected from the group consising of Bacillus, Paenibacillus, Lactobacillus, Brevibacillus, Escherichia, Gluconobacter, Gluconacetobacter, Acetobacter, Streptococcus, Methylobacterium, Pantoea, Pseudomonas, Sphingomonas, Curtobacterium, Knoellia, Massilla, Pedobacter, Skermanella, Clostridia, Klebsiella, Spirillum, Streptomyces, Coniothyrium, Clonostachys, or Achromobacter, Saccharomyces, Hanseniaspora, Trichoderma, Aspergillus, Aureobasidium, Ulocladium, Muscodor, Metarhizium, Beauveria, Paecilomyces, Isaria, and Lecanicillium.

13. The granule of claim 11, wherein the microorganism is Gluconobacter cerinus and/or Hanseniaspora uvarum.

14. The granule of claim 13, wherein the at least one bioactive agent is an enzyme.

15. The granule of claim 14, wherein the enzyme is one or more belonging to any of the classes selection from the group consisting of protease, amylase, cellulase, lipase, mannanase, phytase, fucosidase, oxidase, peroxidase, reductase, transferase and transglutaminase.

16. The granule of any of the preceding aims claim 15, wherein the granule is packaged inside a packaging material with a low water vapor transmission rate.

17. The granule of claim 16, wherein the packaging material having a low water vapor transmission rate is MYLAR®.

18. The granule of claim 17, wherein the granule is packaged together with a desiccant.

19. The granule of claim 18, in which the desiccant is a molecular sieve.
